SEQUENCE LISTING (4 sequences)

SEQ ID NO: 1 - protein, 12 aa, Artificial Sequence
  binding to collagen XII
  DOMAIN (5)..(9)
  DOMAIN (10)..(12)
DLQYWYPIWDTH

SEQ ID NO: 2 - protein, 12 aa, Artificial Sequence
  binding to collagen XII
  DOMAIN (4)..(8)
DAYWHPVWVHDP

SEQ ID NO: 3 - protein, 12 aa, Artificial Sequence
  binding to collagen XII
  DOMAIN (6)..(10)
HVYQKPSYWWYP

SEQ ID NO: 4 - protein, 12 aa, Artificial Sequence
  binding to collagen XII
  DOMAIN (10)..(12)
TWHFVDFSADTH

(19) United States
(12) Patent Application Publication
(10) Pub. No.: US 2023/0026969 A1
(43) Pub. Date: Jan. 26, 2023

(54) Oligopeptide, Testing Kit Thereof And Medical Composition Thereof

(71) Applicant: China Medical University, Taichung (TW)

(72) Inventors: Shih-Chieh Hung, Taichung City (TW); Han-Chung Wu, Taipei City (TW); Chin-Yu Lin, Taichung City (TW); Yi-Hsuan Chi, Tainan City (TW)

(73) Assignee: China Medical University, Taichung (TW)

(21) Appl. No.: 17/781,412

(22) PCT Filed: Dec. 3, 2020
(86) PCT No.: PCT/US20/63007

§ 371 Date: Jun. 1, 2022

Related U.S. Application Data
(60) Provisional application No. 62/942,847, filed on Dec. 3, 2019.

(51) Int. Cl. C07K 17/14; C07K 7/08; C07K 16/18; G01N 33/68

(52) U.S. Cl. CPC C07K 17/14; C07K 7/08; C07K 16/18; G01N 33/6887; A61P 19/02

(57) ABSTRACT

The present disclosure relates to an oligopeptide. The oligopeptide includes an amino acid sequence. The amino acid sequence includes a binding motif, and the binding motif has a specific amino acid sequence. The present disclosure also relates to a testing kit including the oligopeptide and a medical composition including the oligopeptide.

### RELATED APPLICATIONS

This application is a National Stage of International application No. PCT/US2020/063007, filed Dec. 3, 2020, which claims the benefits of priority of U.S. Provisional Application Ser. No. 62/942,847, filed Dec. 3, 2019, which is herein incorporated by reference.

The sequence listing submitted via EFS is incorporated herein by reference. The sequence listing text file submitted via EFS contains the file “CP-4648-US_SequenceListing”, created on May 27, 2022, which is 1,414 bytes in size.

### BACKGROUND

### Technical Field

The present disclosure relates to an oligopeptide, a testing kit medical and a medical composition. More particularly, the present disclosure relates to an oligopeptide specific to collagen XII, a testing kit and a medical composition thereof.

### Description of Related Art

Because articular cartilage lacks the capacity for self-repair, incidences of osteoarthritis (OA) are increasing, especially for those older than 60 years of age. Medication with anti-inflammatory drugs, intra-articular injection with lubricating supplements, and surgeries including microfracture and osaicplasty remain the current modalities for OA treatment, only alleviating symptoms. There are no disease-modifying agents for OA. Cell-based therapy using autologous chondrocyte implantation was only effective in treating focal articular cartilage defects. Transplantation of stem cells or progenitor cells has now emerged as an alternative for chondrocytes in the treatment of OA and osteochondral defects, especially for large lesions.

Mesenchymal stem cells (MSCs), with capacities of self-renewal and multipotent differentiation, are not only used to repair mesenchymal tissues but also used in tissue engineering of cartilage and bone. The long-term safety of intra-articular injection of MSCs has been demonstrated in 41 patients with knee OA. Furthermore, the clinical efficacy and safety of MSC transplantation for OA treatment has been demonstrated in a meta-analysis with 11 eligible trials and 582 knee OA patients.

A two-year follow-up study regarding the efficacy of intra-articular injection of MSCs for the treatment of knee OA revealed potential concerns about the durability of clinical and structural outcomes in low- and intermediate-dose arms of treatment, suggesting the need for further studies. Intra-articular injection of MSCs with hyaluronic acid (HA) as a vehicle showed a superior effect than injection of HA alone for the treatment of OA induced by anterior cruciate ligament (ACL) transection. This study and others revealed that nonspecific binding of MSCs onto the synovium, meniscus, and ligamentous tissues, highlighted the importance of the development of methods for enhancing local delivery of cells to injured articular cartilage. However, there are few, if any, studies focusing on this. Magnetically labeled MSCs have been applied for articular cartilage repair. Although MSCs labeled with magnetic particles exhibit no deterioration in chondrogenic differentiation, there is concern about the uptake of iron by the tissues.

In the current study, we identified OA-targeting peptides through bio-panning of a phage display peptide library with the use of human OA specimens. The OA-targeting peptides were further investigated for application in the delivery of diagnostic agents, lubrication supplements, and MSCs to articular surfaces in an enzyme-induced OA rat model and in an ACL-transection OA swine model.

### SUMMARY

According to one aspect of the present disclosure, an oligopeptide includes an amino acid sequence including a binding motif, wherein:

the binding motif is represented by Formula (i):

WX₁PX₂W (i),

wherein W is tryptophan, P is proline, X₁ and X₂ are respectively an amino acid, and X₁ and X₂ are identical or different from each other; or
the binding motif is represented by Formula (ii):

DTH (ii),

wherein D is aspartic acid, T is threonine, and H is histidine.

According to another aspect of the present disclosure, a testing kit includes the oligopeptide of the aforementioned aspect.

According to further another aspect of the present disclosure, a medical composition includes the oligopeptide of the aforementioned aspect and a treatment molecule or a stem cell binding to the oligopeptide.

### BRIEF DESCRIPTION OF THE DRAWINGS

The present disclosure can be more fully understood by reading the following detailed description of the embodiment, with reference made to the accompanying drawings as follows:

FIG. 1 shows results that intravital imaging demonstrates the binding capability of C5-24 peptide to OA cartilage.

FIG. 2 shows results of application of C5-24 peptide in early OA diagnosis.

FIG. 3 shows results of application of C5-24 peptide in joint lubrication.

FIG. 4 shows results of application of C5-24 peptide in OA regenerative medicine.

FIG. 5 shows results of MRI analysis and Prussian blue staining for MSC tracking.

FIG. 6 shows results of identification of the binding protein of C5-24 peptide.

### DETAILED DESCRIPTION

The present disclosure will be further exemplified by the following specific embodiments to facilitate utilizing and practicing the present disclosure completely by the people skilled in the art without over-interpreting and over-experimenting. However, these practical details are used to describe how to implement the materials and methods of the present disclosure and are not necessary.

I. Results

<Identification of OA-Targeting Peptides>

Using a phage display peptide library, we probed the OA articular cartilage cut from the subchondral bone of knee joints from patients who received total knee joint replacement. The OA cartilage specimens were homogenized for acquiring tissue lysates or cut into square tissue pieces, 5 mm×5 mm in size. Through five rounds of selection of phage-displayed peptides (bio-panning) binding to tissue lysates and tissue pieces, the titers of bound phages significantly increased up to 388-fold, and 864-fold, respectively. Phage clones collected from the fifth round of bio-panning were further subjected to ELISA screening, and clones with high affinity to tissue lysates or pieces were chosen, sequenced, and aligned. Finally, we identified five groups of targeting phages sharing distinctive consensus motifs. The binding abilities of selected phage clones were validated in the human chondrocyte cell line, hPi-GL10, by immunocytofluorescent staining. All of the identified phage clones, labeled with M13-PE (antibody conjugated to fluorescent dye), bound to hPi-GL in a dose-dependent manner. Notably, C5-24 and C5-91 peptides showed specific and remarkable binding scenarios in hPi-GL. To examine phage clones specifically binding to OA cartilage rather than to other soft tissues, such as the synovium and meniscus, the human OA tissue sections were immunostained using horseradish peroxidase (HRP)-labeled phage clones, followed by semi-quantification of the deposited 3, 3-diaminobenzidine (DAB) intensity (− to +++). Particularly, C5-24 (as shown in amino acid sequences of SEQ ID NO. 1) and C5-91 (as shown in amino acid sequences of SEQ ID NO. 2) peptides showed superior binding activity to cartilage, but no binding activity to the meniscus and synovium. Moreover, C5-24 peptide exhibited the best specificity for targeting the territorial region of OA cartilage and was chosen for subsequent studies.

<Intravital Imaging of OA Targeting>

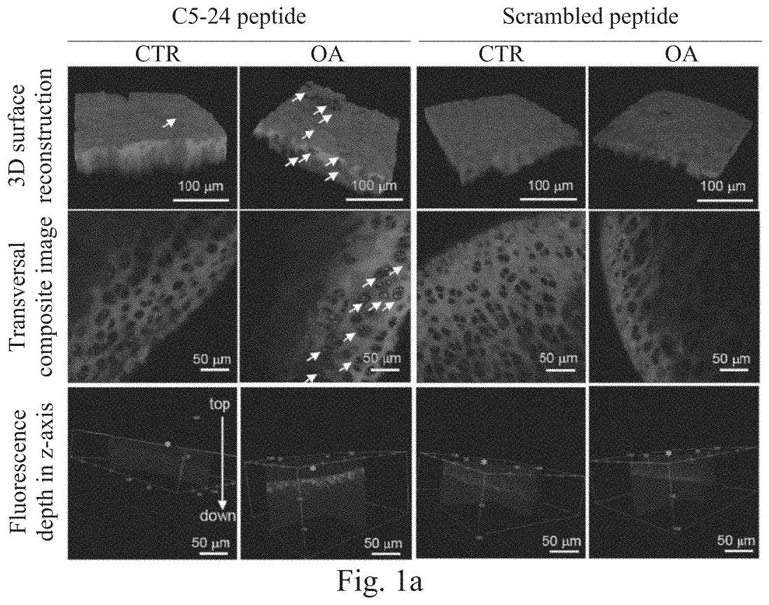

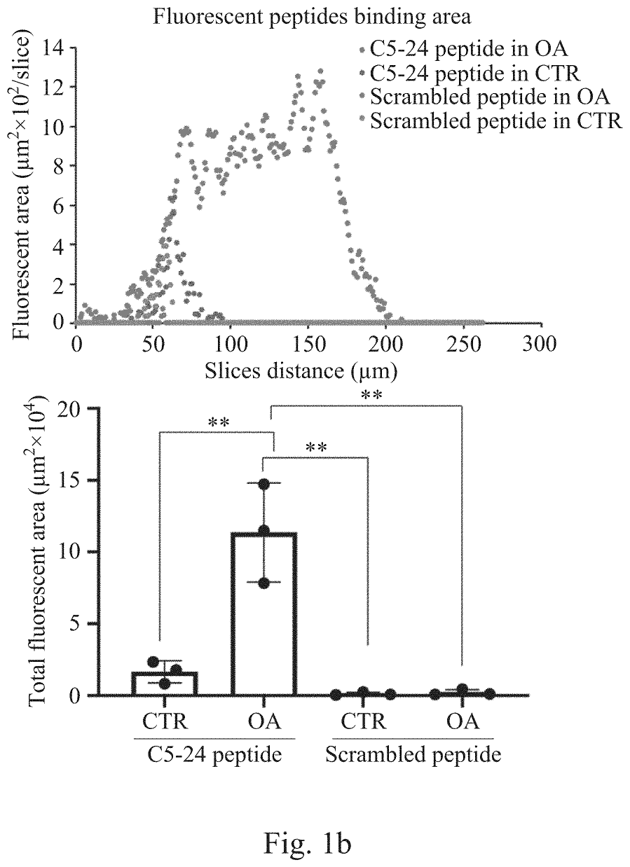

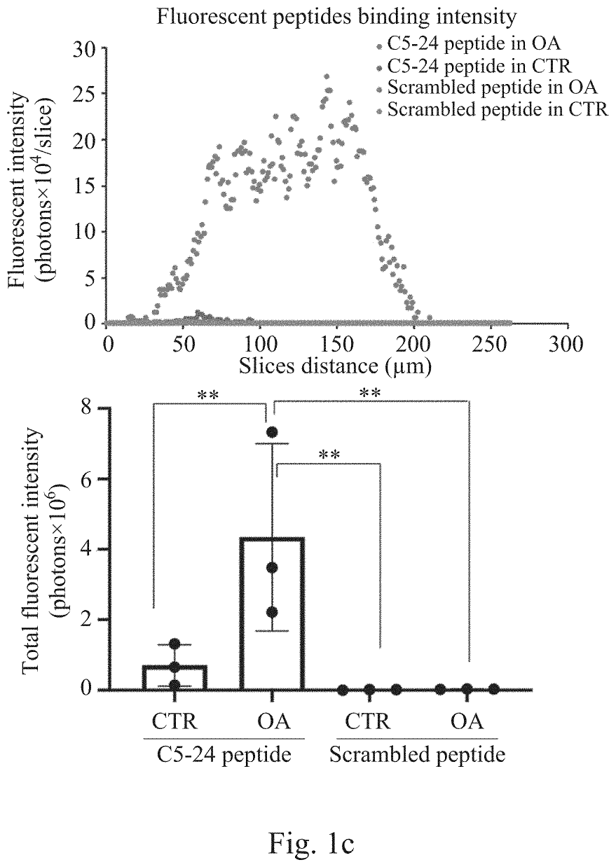

To demonstrate the OA-specific targeting activity of C5-24 peptide, rhodamine-labeled C5-24 peptide and scrambled peptides were separately injected into OA joints in a rat model for two-photon microscopy observation of fluorescence and second harmonic generation (SHG) signals. A scrambled peptide contains all the same amino acids as the original peptide but in new random order. Surface-rendered 3D reconstructed images and transversal composite images of cartilage showed sparse red dots randomly existing in the C5-24 peptide-injected control cartilage, scrambled peptide-injected control cartilage, and scrambled peptide-injected OA cartilage. Conversely, red dots were observed in the C5-24 peptide-injected OA cartilage. When probing type II collagen with SHG, red dots were localized in the SHG signal-free area (FIG. 1a), corresponding to the territorial region of OA cartilage. From the z-axial plane, it is determined that C5-24 peptide reached at least 50 μm in depth in the OA cartilage (FIG. 1a). In addition, the overall fluorescent peptides binding area (FIG. 1b) and binding intensity (FIG. 1c) in all slices were further calculated, showing a significant difference in C5-24 peptide targeting between OA and control cartilage. These data demonstrate the distinguished recognition capability and specificity of C5-24 peptide for targeting the territorial regions of OA cartilage.

<Application in Early OA Diagnosis>

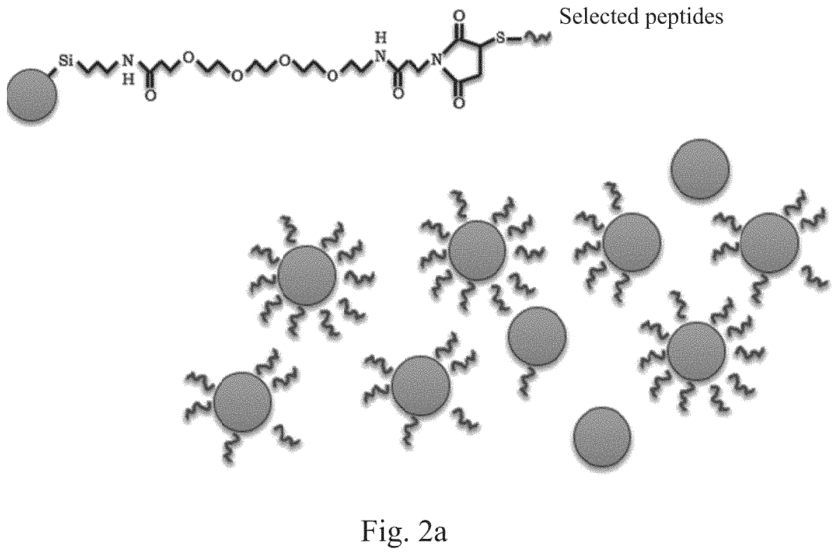

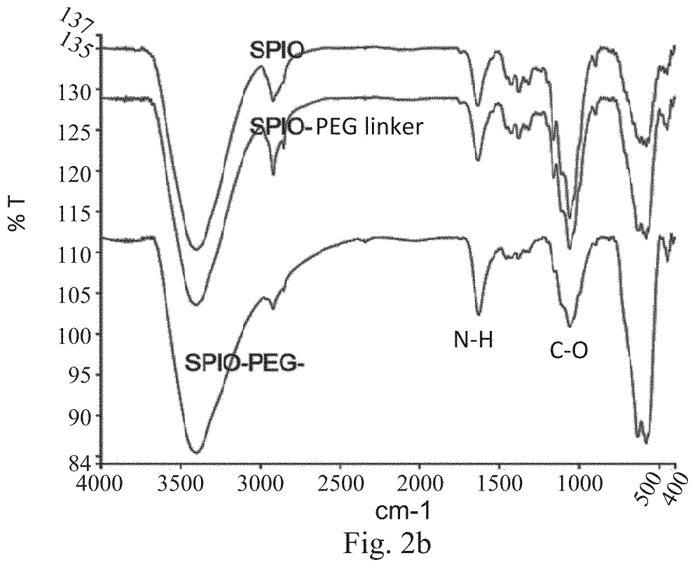

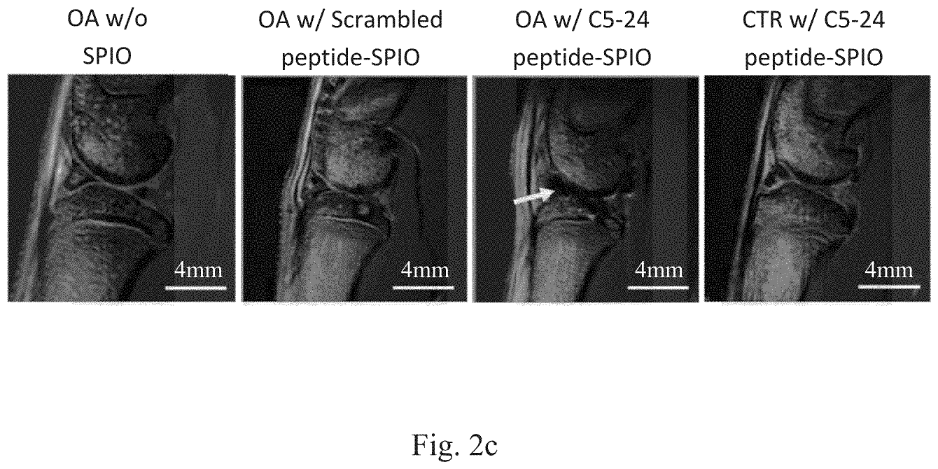

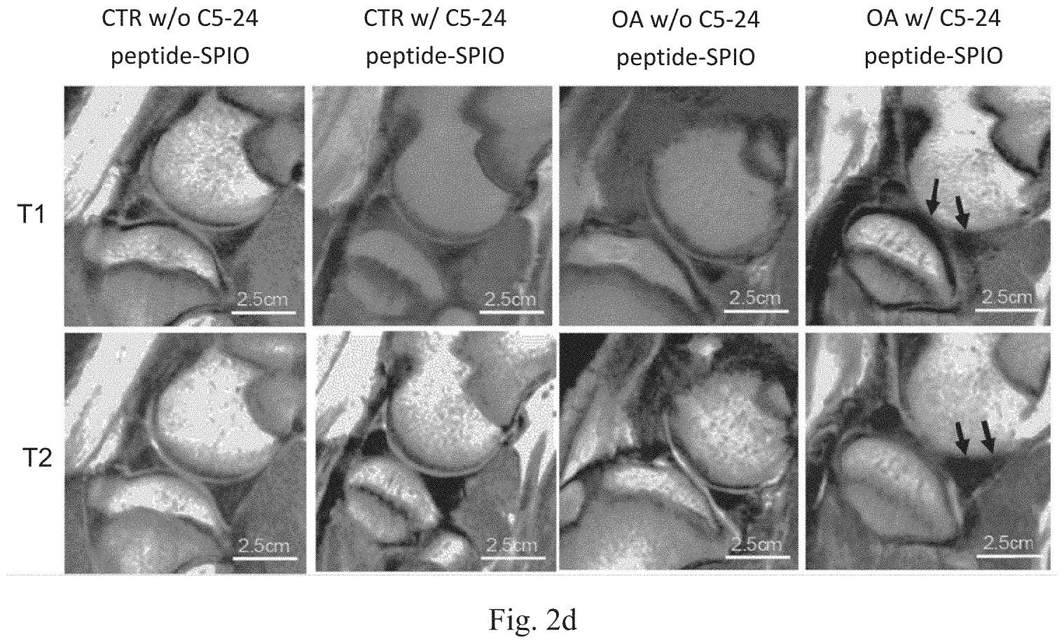

To demonstrate the applicability of OA-targeting peptide in the delivery of diagnostic agents for early diagnosis of OA, C5-24 and scrambled peptide were conjugated with superparamagnetic iron oxide (SPIO) (FIG. 2a). Fourier-transform infrared spectroscopy (FTIR) revealed increased N—H band/C—O stretch ratios, indicating successful installation of SPIO into C5-24 and scrambled peptides (FIG. 2b), which were intra-articularly injected into the OA joints of a rat model established by enzyme digestion. Magnetic resonance imaging (MRI) of the OA knee joints without peptide-conjugated SPIO injection showed no difference compared to the sham control knee joints, revealing the challenge of MRI for early OA diagnosis when the articular cartilage is not severely denuded. Similarly, scrambled peptide-conjugated SPIO that did not bind to the OA cartilage, and the MRI signal reduction also failed to differentiate early OA from sham controls. Conversely, C5-24 peptide-conjugated SPIO bound to OA cartilage and caused MRI signal reduction in OA cartilage but not in healthy cartilage (FIG. 2c). To get one step closer to the clinical setting, the feasibility of C5-24 peptide-conjugated SPIO for early OA diagnosis was further confirmed in a large animal OA model established by ACL-transection in Lanyu minipigs. After 2 months of ACL-transection, the sham control knee joints either with or without receiving C5-24 peptide-conjugated SPIO or the OA knee joints without receiving C5-24 peptide-conjugated SPIO showed no difference in the T1- and T2-weighted MR images (FIG. 2d), indicating the difficulty in using MRI for early OA diagnosis. However, the OA knee that received C5-24 peptide-conjugated SPIO showed enhanced signal reduction in T1- and T2-weighted MR images, demonstrating the sensitivity of C5-24 peptide-conjugated SPIO for early OA diagnosis. Taken together, these data suggest that imaging contrast agents, such as SPIO, when conjugated with C5-24 peptide, could be applied in early OA diagnosis in combination with the MR imaging system.

<Application in Joint Lubrication>

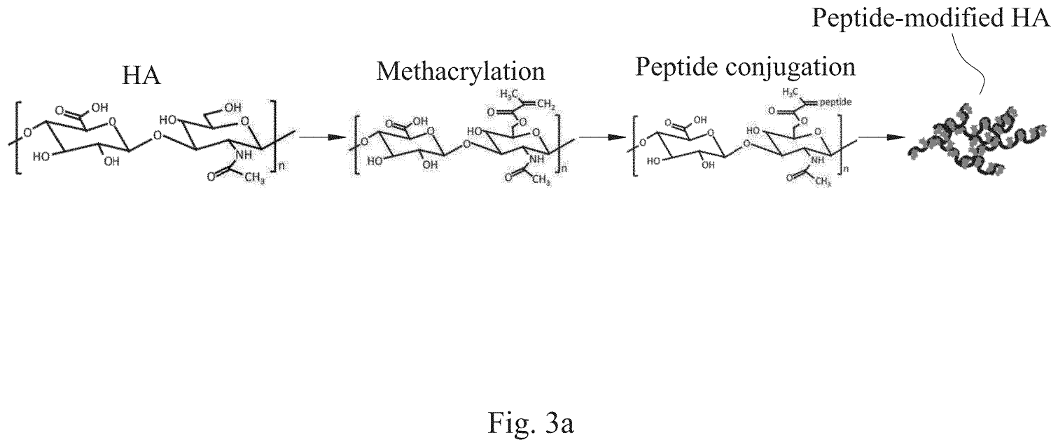

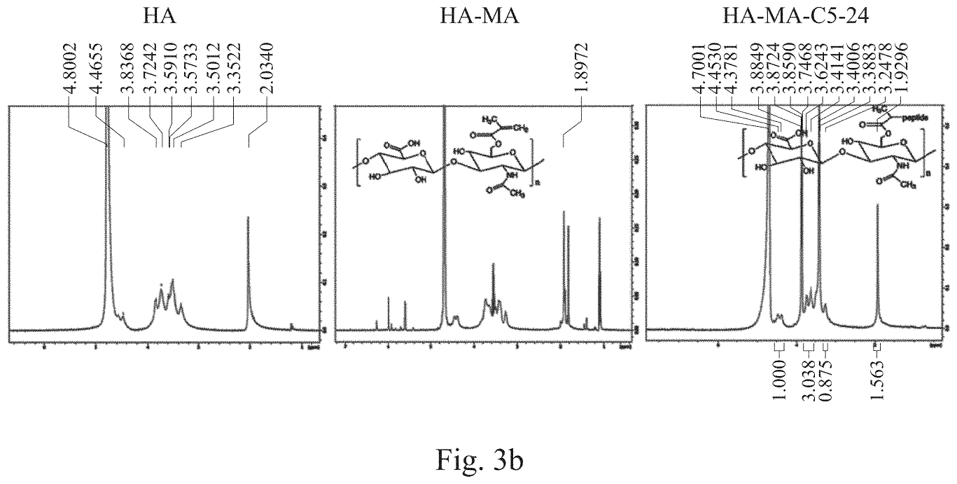

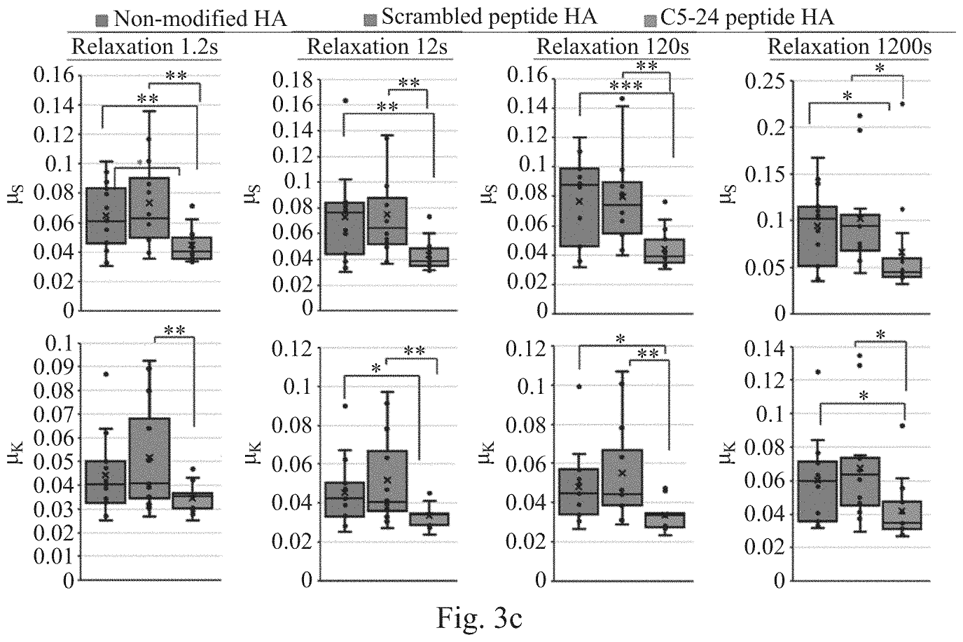

To investigate the potential of C5-24 peptide to deliver HA into OA cartilage for lubrication, C5-24 peptide or scrambled peptides were conjugated with HA, referred to here as C5-24-HA and scrambled-HA, respectively (FIG. 3a). Methacrylation of HA-MA was measured by ¹H proton-NMR (Nuclear Magnetic Resonance) to be about 28.1%, which was used as intermediate product for subsequent C5-24 peptide (FIG. 3b) and scrambled peptides conjugation. The rheological lubrication properties, including static friction coefficient (μₛ) and kinetic friction coefficient (μₖ), were assessed using a rotational test protocol modified from a previous report, and compared among paired human OA cartilage cylinder discs (collected from 13 individuals) treated with non-modified HA, scrambled-HA, or C5-24-HA. The total friction coefficients for non-modified HA, scrambled-HA, and C5-24-HA in 1.2 s relaxation scenario were: 0.065, 0.073, and 0.044 in μₛ and 0.045, 0.052, and 0.034 in μₖ, respectively, with 32.3% and 24.4% reduction in C5-24-HA compared to non-modified HA; in 12 s relaxation scenario were: 0.072, 0.075, and 0.043 in μₛ and 0.045, 0.052, and 0.033 in μₖ, respectively, with 40.3% and 26.7% reduction in C5-24-HA compared to non-modified HA; in 120 s relaxation scenario were: 0.077, 0.079, and 0.044 in μₛ and 0.048, 0.055, and 0.034 in μₖ, respectively, with 42.9% and 29.2% reduction in C5-24-HA compared to non-modified HA; and in 1200 s relaxation scenario were: 0.094, 0.102, and 0.066 in μₛ and 0.060, 0.067, and 0.042 in μₖ, respectively, with 29.8% and 30% reduction in C5-24-HA compared to non-modified HA (FIG. 3c). Briefly, C5-24-HA showed statistically significant superior static and kinetic friction characteristics in comparison with non-modified HA and scrambled-HA in all relaxation stages, demonstrating superior lubrication. Moreover, C5-24-HA exhibited better lubrication than non-modified HA and scrambled-HA in the rheological pre-condition stage and torque measurements. Representative individual patient data are placed in the supplementary information, showing the same scenario with the gradual loss of cartilage disc height in the 3600 s relaxation time in the pre-conditioning stage, but returning to consistent cartilage disc height in the following four stages of the relaxation period, which reduced the factors affecting the friction measurement. Together, these data suggest the applicability of C5-24 peptide in the development of novel and effective joint lubricants for OA.

<Application in OA Regenerative Medicine>

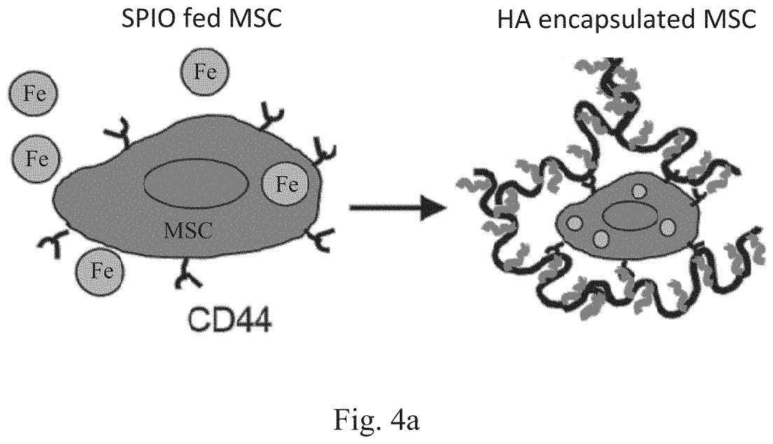

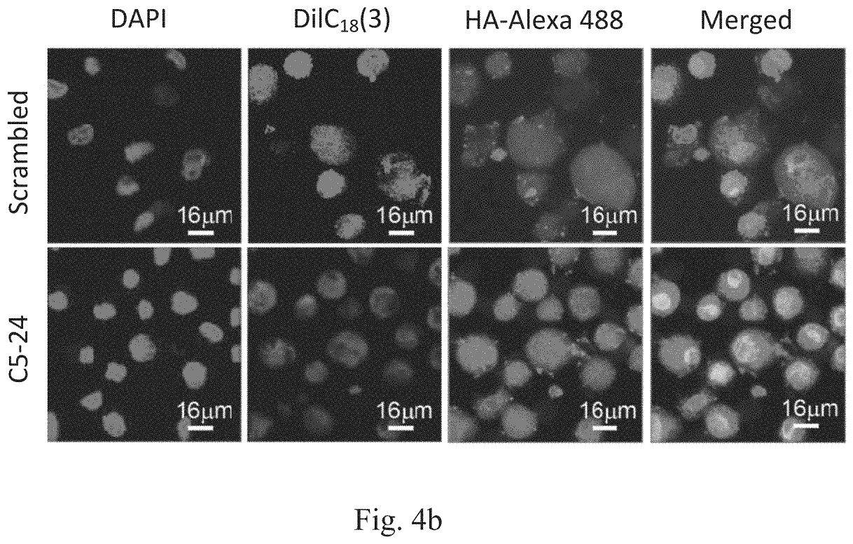

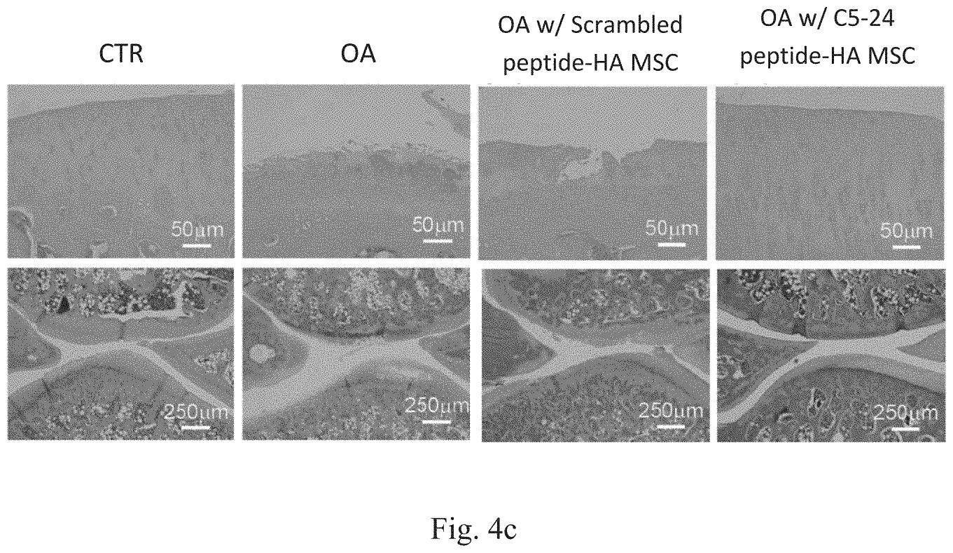

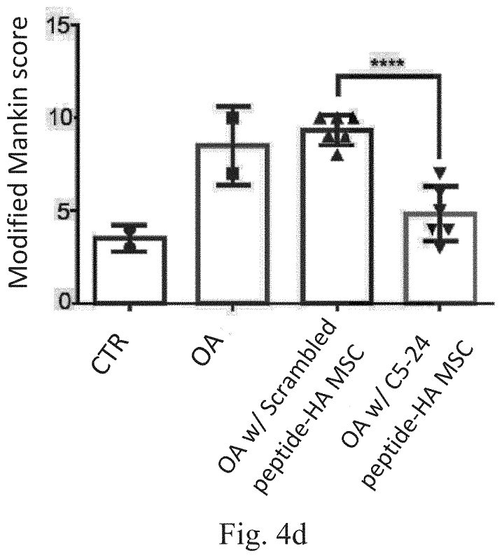

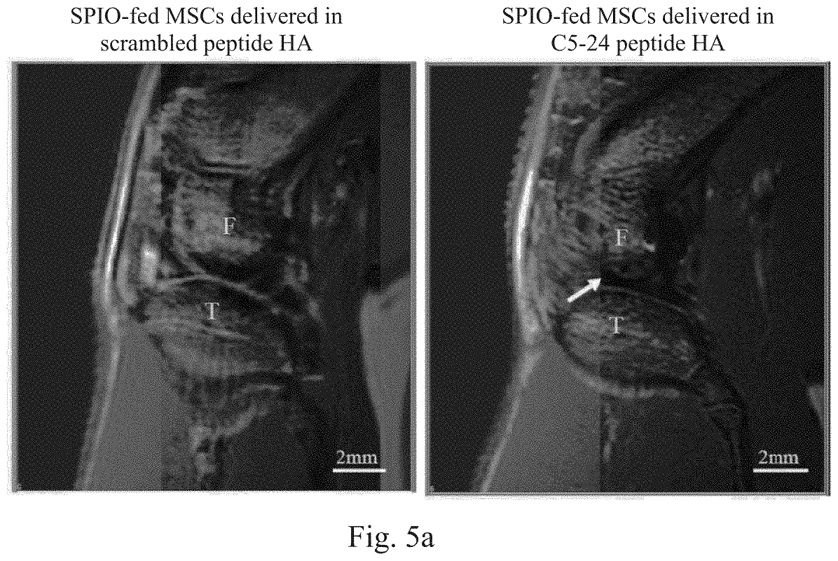

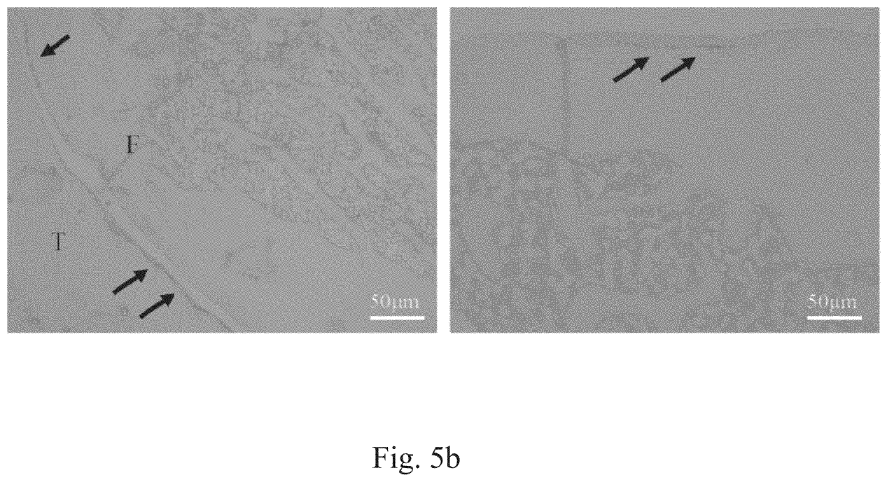

C5-24-HA may be applied to MSC regenerative medicine by binding to CD44, the HA receptor, which is extensively expressed on the MSC cell surface, and delivering MSCs to the OA cartilage surface. Moreover, the chondrogenic activity of HA is likely to induce MSC chondrogenesis, as demonstrated previously. To demonstrate this, rat MSCs were fed with SPIO for subsequent tracking and incubated with fluorescent-conjugated C5-24-HA or scrambled-HA (FIG. 4a). Fluorescence microscopic observation demonstrated that MSCs were tightly surrounded by green fluorescence (FIG. 4b, shown in a black and white schema). Furthermore, after incubation with C5-24-HA or scrambled-HA, MSCs were immediately injected into OA joints in a rat model, and the joints were subjected to histological examination 8 weeks post-transplantation. Histomorphometric analysis revealed the successful induction of OA when compared OA group with sham control group (FIGS. 4c, 4d). Moreover, knee joints receiving MSCs delivered by C5-24-HA had apparent cartilage regeneration and safranin-O staining (FIG. 4c), while those receiving MSCs delivered by scrambled-HA still exhibited severe OA, showing multiple cracks on the surface of the cartilage with the loss of safranin-O staining. Quantification of OA degree by modified Mankin score also revealed that the former had better improvement in OA than the latter (FIG. 4d). For cell tracking of SPIO-fed MSCs transplanted into the OA joint, rats were subjected to MRI scanning (FIG. 5a) and Prussian blue staining (FIG. 5b), 3 days post-transplantation, revealing specific homing of MSCs to OA cartilage in the C5-24-HA-assisted group, but not in the scrambled-HA-assisted group. These data suggest the applicability of C5-24-HA for enhancing MSC regenerative medicine.

<Identification of Binding Proteins>

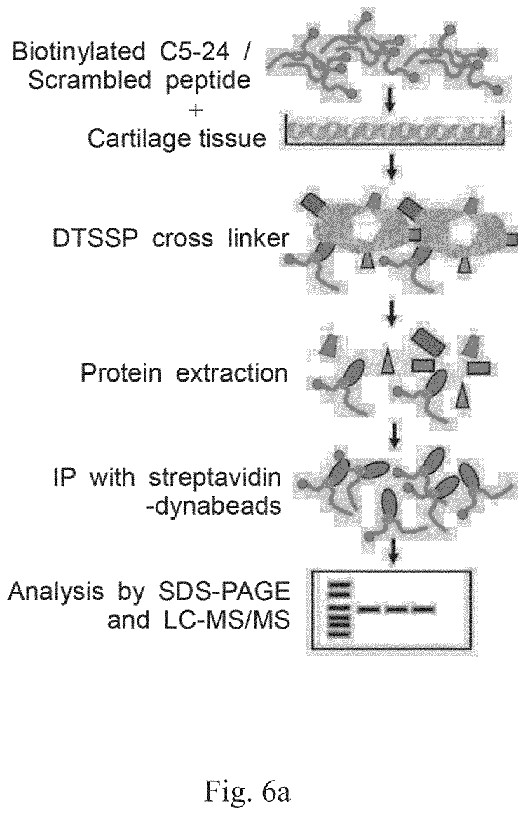

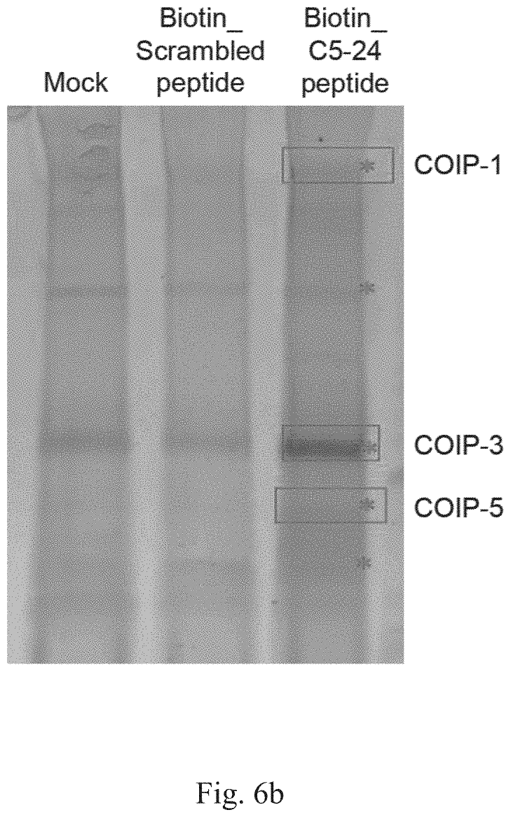

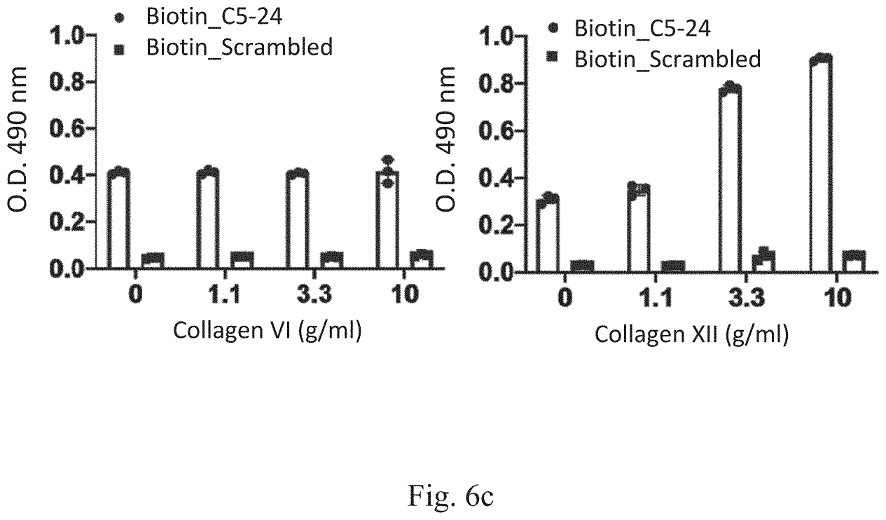

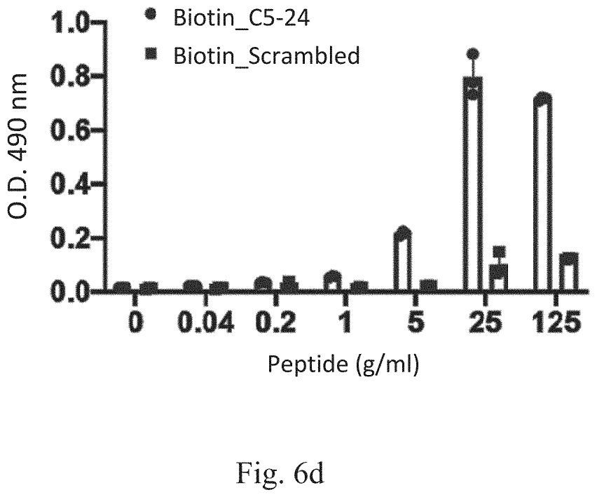

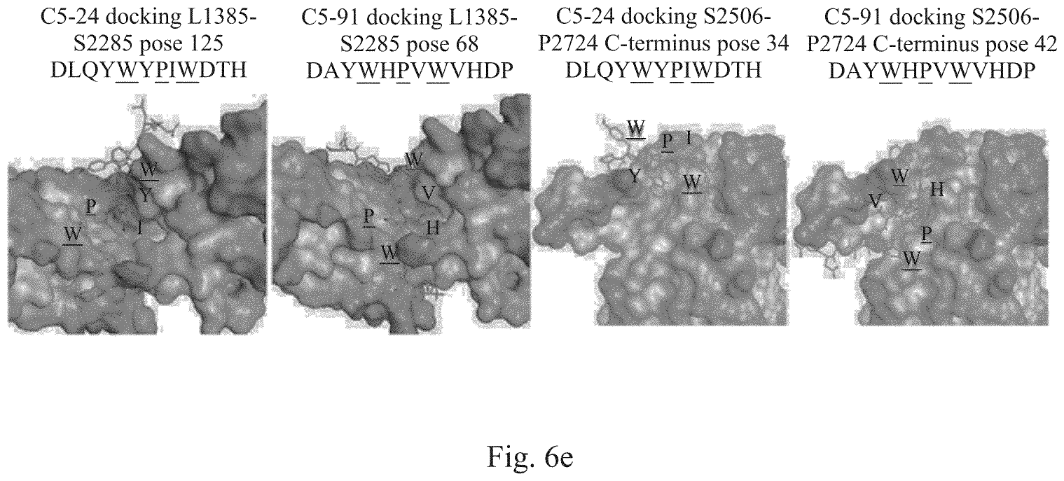

To identify the putative target protein derived from human OA cartilage tissue that binds to C5-24 peptide, we used biotinylated C5-24 peptide combined with a chemical cross-linker, 3,3′-Dithiobis(sulfosuccinimidylpropionate) (DTSSP), and subsequent sodium dodecyl sulfate polyacrylamide gel electrophoresis (SDS-PAGE) and liquid chromatography with tandem mass spectrometry (LC-MS/MS) to identify the binding target (FIG. 6a). Silver staining revealed several sharp bands, such as coimmunoprecipitated proteins COIP-1, COIP-3, and COIP-5 (FIG. 6b), which were separately collected, digested with trypsin, and analyzed by LC-MS/MS. The fragments were identified by searching the Swiss Protein Database through algorithms using MASCOT and TurboSequest search engines. We found several candidate proteins, including collagen alpha-1 (XII) and collagen alpha-3 (VI) fragments with a probability score, indicating the probability of that peptide belongs to a protein, of up to 850 and 372, respectively, higher than most of the other identified peptides. To further confirm these protein fragments as the target protein of the C5-24 peptide, we examined the mutual binding activities between the target protein and biotinylated C5-24 peptide using ELISA. We first pre-coated the ELISA plate with a specific collagen concentration, to identify the optimal collagen concentration for peptide binding (FIG. 6c), and subsequently used the collagen alpha-1 (XII) at 3.3 μg/mL to examine peptide binding (FIG. 6d). We found that biotin-C5-24 peptide bound to collagen alpha-1 (XII) and collagen alpha-3 (VI) fragments, but biotin-scrambled peptides did not. However, a mutual dose-dependent binding was observed only between collagen alpha-1 (XII) and biotin-C5-24 peptide (FIGS. 6c, 6d). In addition, there was no difference in the binding of biotin-C5-24 peptide and biotin-scrambled peptide to Bovine Serum Albumin (BSA). Together, these data suggest that collagen alpha-1 (XII) is the target protein of the C5-24 peptide.

To predict the structure of the protein-peptide complex, the protein-peptide docking was approached through homology modeling and the establishment of several reliable structure models targeting human collagen XII, based on searching for sequence 2 similarities. These structural models were subsequently applied to calculate the possible molecular docking poses with C5-24 and C5-91 peptide chains, which were both the most promising peptide chains and could be selected for further experiments in our study. The protein-peptide docking models were mainly based on an algorithm in compliance with the lowest Gibbs free energy and chemical thermodynamics, after peptide chain binding with the target protein. Our data showed that both C5-24 and C5-91 peptide chains were targeted to the pocket site of collagen XII in the region of L1385-S2285 with pose 125 and pose 68, and targeted to the C-terminus of S2506-P2724 with pose 34 and 42, respectively, which share identical docking sites for C5-24 and C5-91 with the highest pose frequencies (FIG. 6e). Furthermore, these predicted poses share the important consensus binding motifs, WXPXW, which may dominate the major docking affinity between peptide chains and collagen XII. In addition, the sequence homology of collagen XII between humans, swine, rabbits, rats, and mice reached 90.3% similarity and 83.7% identity, and phylogenic analysis revealed a high genetic correlation of Collagen XII between these five species. C5-24 peptide was highly reliable in rodent, rabbit, and swine OA model examinations. Moreover, according to the consensus domains in Table 1, the peptide sequences in the same groups shared important and identical motifs, such as FVEW and DTH in groups 1 and 3, respectively.

TABLE 1

| | Phage clone | Amino acid sequence | Frequency |
|---|---|---|---|
| 1 | C5-3, C5-50, | G D Y V I D W N F I E W | 4/24 |
| | C5-84, C5-87 | | |
| | C5-37 | T V G S F F V E W M M H | 1/24 |
| | C5-66 | D I G G W F V E W S L A | 1/24 |
| 2 | C5-22, C5-83, | D W G Y F S W A Y D S A | 3/24 |
| | C5-92 | | |
| | C5-43 | D W Y T V S W L T D S N | 1/24 |
| 3 | C5-42, C5-91 | D A Y W H P V W V H D P | 2/24 |
| | C5-24 | D L Q Y W Y P I W D T H | 1/24 |
| | C5-12 | H V Y Q K P S Y W W Y P | 1/24 |
| | C5-21 | T W H F V D F S A D T H | 1/24 |
| 4 | E5-8, E5-48 | D Y F T L D F T F D S W | 2/24 |
| | C5-46 | N Q V Y F H Y F D L D F | 1/24 |
| 5 | E4-14, E5-24 | S P W W L W K A H N E A | 2/24 |
| 6 | C5-38 | E V F N H Y I Q Y S T E | 1/24 |
| | E4-1 | L P G M E L F W N V A N | 1/24 |
| | E4-4 | D T F V F G S S K W R A | 1/24 |
| | E4-15 | S N N M R A P V N E I Y | 1/24 |

Finally, we demonstrated the exclusive expression of collagen XII in OA articular cartilage. The expression of collagen XII was only observed in rat OA cartilage, but not in normal articular cartilage. In addition, the expression of collagen XII was only observed in human OA cartilage, but not in non-OA cartilage. Consistent with the area where C5-24 peptide binds (FIG. 1a), collagen XII is mainly expressed in the territorial regions of clustered chondrocytes. These data are also supported by a cohort study that included 161 OA patients and 29 non-OA patients. The preliminary analysis revealed a significant increase in COL12A1 mRNA levels in combined OA hip and OA knee cartilage relative to the non-OA cartilage.

<Application in Disease-Modifying OA Drugs (DMOADs)>

No drugs are currently approved DMOADs. OA therefore can be a serious disease with an unmet medical need for therapies that modify its underlying pathophysiology and translate to long-term, clinically relevant benefits. Currently, there are several drugs in phases II OR III or in the preclinical stage, including fibroblast growth factor-18 (Sprifermin) targeting cartilage regeneration, and Kartogenin that promotes the dissociation and nucleus internalization of core binding factor beta (CBFP) and stimulates cascaded chondrogenesis. All of these developing DMOADs could be further assisted by OA-targeted peptides developed in this study to accelerate delivery to OA tissues. Besides, most of these drugs are focused on an intra-articular route of administration as opposed to systemic pharmacotherapy, aiming to enhance the local bioavailability of drugs and bypass conventional barriers, and to minimize systemic toxicity and enhance safety profile by reducing off-target effects. It is however, important to recognize the marked placebo effect from local intra-articular administration, making the assessment of efficacy more challenging. Improvements in the precision of technology applied to deliver therapeutic agents to OA sites, such as the peptide discovered in this study may lead to the successful development of effective therapies for OA. Future efforts should be directed at delivering disease-modifying drugs in a sophisticated carrier equipped with an OA-targeting peptide to enhance the development of DMOADs.

We have identified several phage-encoded peptide motifs (WXPXW and DTH) that home selectively to an OA joint without any significant targeting to other articular soft tissues, including synovial tissues, meniscus, and ligaments. Moreover, we identified C5-24 and C5-91 peptides that specifically bind to the territorial region of chondrocytes in OA joints. C5-24 has been successfully conjugated to SPIO and HA for OA diagnosis and lubrication purposes, respectively. Although the C5-91 peptide has not been confirmed to deliver diagnostic agents or lubricants to the articular surface in OA joints, C5-91 peptide was considered to have the same function since it has the same size and shares the same motif as C5-24 peptide.

Although collagen II is the basis for hyaline cartilage, making up 85-90% of all protein in articular cartilage, aging or OA leads to its damage, starting around chondrocytes (territorial region) at the articular surface, and extending into the whole cartilage with progressive degeneration. Given that collagen II is not specifically expressed in OA, collagen II-targeting peptides may not be applied in OA diagnostics, therapeutics, lubrication, and regenerative medicine. In contrast, OA-targeting peptides sharing the binding motif WXPXW were experimentally and in silico demonstrated to home selectively to territorial regions and bind to collagen XII that is exclusively expressed in OA cartilage as demonstrated in the current study. Immunofluorescence studies with the antibody demonstrated that collagen XII is localized in collagen I-containing dense connective tissue structures such as tendons, ligaments, perichondrium, and periosteum in embryonic tissues, suggesting its emergence during articular joint degeneration and regeneration (type XII collagen also expresses in tissues of cornea, intervertebral disc and trachea). Further studies are needed to clarify the role of collagen XII in OA regeneration. In conclusion, we developed a novel delivery platform targeting collagen XII for the improvement of OA lubrication, diagnostics, treatment, and regenerative medicine.

Although peptides that bind to collagen XII were developed for diagnosis, lubricant and regenerative medicine in OA, they may be suitable for other diseases, such as corneal ulcers and perforations that may occur in severe dry eye, or injuries or degenerative diseases involving other tissues containing hyaline cartilage, such as intervertebral discs and tracheal cartilage. For example, collagen XII, expressed in Bowman's layer of cornea, is overexpressed during corneal ulcer and scar formation, therefore the functionalized collagen XII targeting peptides can help the delivery of lubricants, anti-inflammatory drugs and stem cells for the treatment of corneal ulcers. In conclusion, we developed a novel delivery platform targeting collagen XII for improving OA lubrication, diagnosis, treatment, and regenerative medicine. The platform can also be used to treat other diseases, such as eye ulcers and diseases involving other tissues containing hyaline cartilage.

<Materials and Methods>

The aforementioned embodiments of the present application are performed based on the following methods and materials, and the details thereof are described as follows.

<Preparation of Cartilage Specimens for Bio-Panning and ELISA Screening>

To avoid interference from individual differences among patients, we used surgical articular cartilage specimens from the same OA patient for the five rounds of bio-panning in a phage display experiment. The following processes were used to ensure that the particle-size composition of the cartilage used for the five rounds of bio-panning was consistent. A human surgical OA specimen weighed 3.2 g was added to two volumes of phosphate-buffered saline (PBS) and homogenized. The cartilage homogenate was centrifuged at 800×g and 4° C. for 10 mins, and the precipitate was collected as “the large particle cartilage sample (C1)”. The supernatant was added to a new centrifuge tube, followed by centrifugation at 1,500×g and 4° C. for 10 mins, and the pellet was collected as “the cartilage sample with medium particles (C2)”. Following centrifugation of the supernatant again at 2,000×g and 4° C. for 10 mins, the precipitate was collected as “the small-particle cartilage sample (C3)”. The supernatant at this time was separately collected as the “cartilage tissue lysate” for another five rounds of bio-panning, which was different from the bio-panning performed on the “cartilage tissue pieces”. For “cartilage tissue lysate” bio-panning, C1, C2, and C3 were weighed and aliquoted into five equal parts, respectively. Each round of bio-panning used a mixture of an aliquot of the C1, C2 and C3, for five rounds.

For “cartilage tissue pieces” bio-panning, the cartilage specimens were also cut into square pieces (5×5 mm in size) and adhered to a 96-well ELISA plate with nail polish, one piece per well, for the chondrocyte binding screening.

<Bio-Panning of Phage Clones Targeting OA Cartilage Tissue Lysate and Pieces>

For “cartilage tissue lysate” bio-panning, the tissue lysate supernatant was diluted ten-fold with coating buffer [0.1 M NaHCO₃, pH 8.6] and coated fresh on 10-cm Petri dishes for bio-panning (and 96-well ELISA plates for screening) at 4° C. for 24 hours before use. The tissue lysate-coated plate was blocked with 1% BSA in PBS at 4° C. overnight, 10 pfu of the Ph.D.-12™ phage (New England BioLabs, Ipswich, Mass., USA) display peptide library was added and incubated at 4° C. for 1 hour. After washing, the bound phages were eluted with 1 ml of the log-phase ER2738 culture at 37° C. with 100 rpm shaking for 20 mins. This eluted phage pool was amplified and titrated in an ER2738 overnight culture. The recovered phages were used as input for the next round of panning, and total 130 phage clones were randomly selected from the fifth round of bio-panning to be cultured for ELISA screening.

The processed cartilage specimen was blocked with 1% bovine serum albumin (BSA) in PBS at 4° C. for 1 hour for each round of “cartilage tissue pieces” bio-panning. The Ph.D.-12™ (New England BioLabs, Ipswich, Mass., USA) phage display peptide library, which initially contained 10 plaque-forming units (pfu), was added and incubated at 4° C. for 1 hour. After washing, the bound phages were eluted with 1 ml of log-phase Escherichia coli ER2738 culture (New England BioLabs) at 37° C. with 100 rpm shaking for 30 mins. This eluted phage pool was amplified and titrated in an ER2738 overnight culture. The recovered phages were used as input for the next round of panning, and total 95 phage clones were randomly selected from the fifth round of bio-panning to be cultured for ELISA screening.

<Identification of Amino Acid Sequence Motifs to Target OA Cartilage>

The binding activity of the selected phage clones to cartilage tissue lysate and cartilage tissue pieces was examined by ELISA. Phage clones with the highest binding affinity (A490 value>0.15 for the cartilage tissue lysate and A490 value>2.0 for the cartilage tissue piece) were selected and sequenced. We identified five distinct groups with differing consensus motifs by amino acid sequence alignment (as shown in Table 1).

<Validation of Peptides Targeting OA Cartilage Using Immunofluorescence of hPi-GL Chondrocyte Cell Line>

For examination of those phage clones, slide-cultured hPi-GL cells were fixed with 4% paraformaldehyde in PBS at room temperature for 15 mins, washed with PBS and permeabilized with 0.1% Triton X-100 at room temperature for 30 mins, blocked for nonspecific binding with 1% BSA/PBST. The slide-cultured hPi-GL cells were separately incubated with 4×10⁸ pfu, 8×10⁸ pfu, and 109 pfu selected phage clones at 4° C. for 1 hour. After removing the unbound phages by washing, the cells were incubated with anti-M13 mouse mAb (GE Healthcare, Milwaukee, Wis., USA) as the primary antibody and R-Phycoerythrin-AffiniPure F(ab′)2 fragment goat anti-mouse IgG (Jackson ImmunoResearch Inc.) as the secondary antibody at room temperature for 1 hour, respectively. Then, washed with PBST, and counterstained with Hoechst 33258 (1 μg/ml; Sigma-Aldrich) at room temperature for 10 mins. The cells were analyzed for phage binding and localization by fluorescence using confocal microscopy (Zeiss LSM 700).

<Selection of Peptides Targeting OA Cartilage but not Synovium and Meniscus>

To examine localization of the targeting phages bound to articular tissues, human OA cartilage specimens were used for examination. Paraffin-embedded human OA tissue, synovium, and meniscus sections were retrieved from specimens of human OA surgical treatment under the approval of the Institutional Review Board of China Medical University Hospital (IRB no. CMUH108-REC1-046 and T-CMU-23728). Written informed consent was obtained, and all human tissue samples were coded for anonymity. All sections were dried, deparaffinized and rehydrated by standard protocols, and subsequently incubated with C5-87, C5-66, C5-83, C5-91, C5-24, E5-8, and C5-46 phage clones, or the control phage (5×10⁸ pfu/μl). After washing, the sections were treated with anti-M13 mouse mAb (GE Healthcare) for 1 hour at room temperature. Following several washing steps, a biotin-free super sensitive polymer-HRP detection system (Biogenex, Fremont, Calif., USA) was used to detect immunoreactivity. The slides were lightly counterstained with hematoxylin, mounted with Aquatex (Merck, Darmstadt, Germany), and examined by light microscopy. Peptide sequences displayed on the phage clones that exhibited prominent binding to chondrocytes, but not synovium or the meniscus, were selected and synthesized for subsequent studies.

<Rat OA Model Establishment>

The OA in rat model was established as previous described with slightly modification. In brief, the male SD rats≅300 grams in weight were used in this study. All animal experiments were approved by the China Medical University Committee for the Use and Care of Animals. Rats were kept under standard laboratory conditions (temperature 24° C., 12 h light-dark cycle), fed standard diet and drank tap water. Rats were anesthetized with 2.5% isofluorane (Abott, USA) in 70 ml/min flow rate before every injection. Rat joint OA was induced in the right knees in each group by injecting 0.2 ml of 4% papain solution (Sigma-Aldrich, USA) with 0.1 ml of 0.03 M cysteine (Sigma-Aldrich, USA) as activator. Same amount of saline was injected into the left knees in each group. Injection was repeated on the fourth and seventh days, respectively, and two weeks after the last papain injection, rat knees were removed for histological analysis to confirm the formation of OA. The established OA model in rats was further used in the following experiments for intraarticular injection.

<Preparation of Rhodamine Labeled C5-24 Peptide and 2-Photon Microscopic Observation>

To demonstrate the OA specific targeting activity of C5-24 peptide, the DYLWQYPDITWH peptides, which is not able to bind to OA cartilage, was used as scrambled peptides. The rhodamine-labeled C5-24 and scrambled peptides were separately injected into rat joints without (control) or with enzyme-induced OA. The C5-24 and scrambled peptides were chemically synthesized as accordingly (ABI, USA), modified with Biotin-PEG2-Iodoacetyl bridge linker (Thermo Fisher Scientific, USA) in HEPES buffer pH 8.0 through click reaction, further linked with avidin labeled rhodamine (Jacksonlmmuno, USA) and subjected to dialysis in ddH₂O in M.W. 4K cut-off to remove the unlabeled rhodamine, further lyophilized and stored in −20° C. Aliquots of 1 μg rhodamine-labeled peptides in 40 μl PBS were used for intraarticular injection with using 30 G syringes.

Rat Knees were removed at 1-day post-injection, and both femoral condyles and tibias were cleaned thoroughly, immersed in PBS and sophisticatedly attached on 3.5 cm dish for 2-photon microscopic observation. The microscope system was operated using a near-infrared femtosecond laser (Mira 900, Coherent, USA) at the central wavelength of 810 nm, 76 MHz pulse repetition rate, and 200 fs pulse width for imaging. The laser power was controlled to 20 mW that is sufficient to produce SHG and TPEF, and also prevented photodamage during continuous illumination. Thus, the wavelength of SHG from collagen fibers is 405 nm, while the TPEF from collagen, elastin, FAD, and NADH is approximately ranging from 450 to 650 nm. All images were obtained by a laser scanning unit (Fluoview 300, Olympus, Japan), a pair of two objective lenses for both lasers focusing and collection of photons (UPlanSApo 20×/0.75, Olympus, Japan), and two photomultiplier tubes respectively for SHG and TPEF detection (R3896, Hamamatsu, Japan). SHG and TPEF were filtered from the intense excitation laser background by a combination of band-pass filter (FF01-405/10, Semrock, USA) and color glass (BG39, Schott, Germany). And, then splitted by a dichroic mirror (FF435-Di01, Semrock, USA) and forward detected. Note that we used a cube polarizing beam-splitter (GT10-B, Thorlabs, USA) combined with a half (AHWP05M-980, Thorlabs, USA) and a quarter (AQWP05M-980, Thorlabs, USA) waveplates to demonstrate LP and CP imaging, respectively. Only the extinction ratio of linear polarization larger than 50:1 and the ellipticity of circular polarization (Imax/Imin) less than 1.1 after the focusing objective lens can then be used for the following two-photon imaging. The acquired images were mainly processed and analyzed with ImageJ/FiJi software (National Institutes of Health, Bethesda, Md., USA). The type II collagen structure reconstructed through the second harmonic generation images (FIG. 1a) showed porous collagen fiber inter-connected structures (green color) surrounding nested chondrocytes (black area).

<Preparation of C5-24 Peptide-Conjugated Superparamagnetic Iron Oxide (SPIO) and IR Spectroscopy>

The C5-24 and scrambled peptides were chemically synthesized, meanwhile aminosilane modified SPIO particles in 50 nm in diameter (Chemicell GmBH, Germany) were firstly crosslinked with succinimidyl-[(N-maleimidopropionamido)-tetraethyleneglycol] ester (Thermo Fisher Scientific, USA) in sodium bicarbonate buffer pH 8.5 to form amide bonds, and subsequently interacted with sulfhydryl group on cysteine of the peptides in pH 7.2 to form a stable thioether bond and subjected to dialysis in ddH₂O in M.W. 10K cut-off to remove the free-forms of peptides, bridge linkers and salts, leaving groups and further concentrated in reduced pressure, resuspended in PBS and stored in 4° C. for experiments not longer than 2 weeks. To analyze the installation of peptides on SPIO, a part of prepared SPIOs were lyophilized, grounded with potassium bromide (KBr) thoroughly in 1:100 wt./wt. and compressed in 200 pound/inch² to form a thin pellet for further infrared radiation spectroscopic analysis (Perkin Elmer, USA). IR was scanned from 400-4000 1/cm frequency to record the characteristic-group and finger-print region molecular groups in transmission mode, respectively.

<Magnetic Resonance Imaging (MRI) Analysis of OA in Rat Model>

Rats at the indicated time points as results shown were anesthetized by inhalation and subjected to MRI scanning. MRI scans were performed using a 4.7T MR scanning system (Bruker BioSpin, Germany) at the Institute of Biomedical Sciences, Academia Sinica in Taiwan. T1-weighted and T2-weighted sagittal sections were rendered using the following settings: fast spin echo sequence with a time to repetition of 2000 ms and time to echo of 72 ms; slice thickness was 1 mm; interslice gap 1 mm; matrix 256; TE 60; TR 2000; field of view 60 mm; number of averages 2. A 60 mm volume resonator and a 2 cm diameter surface receive coil were used to maximize image resolution and quality. Tomographs DICOMs of MRI were analyzed by Osirix MD (Osirix Ltd., USA).

<OA in Mini-Pig Model, Intraarticular Injection of C5-24 Eptide-Conjugated SPIO and 3T-MRI Analysis>

For surgery of anterior crucial ligament (ACL) transection to establish OA, a Taiwan Lan-Yu minipig (9-month-old, weight≈50-60 kg) was anesthetized by combined intramuscular (i.m.) injection of Stresnil (20 mg/kg) and atropine sulfate (0.02 mg/kg), followed by i.m. injection of Zoletil® 50 (4 mg/kg, Virbac Animal Health, France) 15 mins later. In order to get a more homogenous group of knee joints, only female pigs were included in the current study. During the surgery, the animals continued to be anesthetized with gas containing oxygen (flow rate of 1.5 L/min), nitrous oxide (flow rate of 1 L/min) and 1% isoflurane. The right rear limb was washed and covered sterilely. Following intravenous administration of cefazolin (2 g), an incision in the skin of approximately seven cm was made in the right knee from the patella to the tuberositas tibiae. The joint was then opened medial to the patellar ligament and the patella is partly luxated. The ACL was then fixed by a clamp and cut at the distal end using a scalpel. To avoid spontaneous healing of the ACL after this transection, a proximal resection was additionally carried out using an electrical arthrosector. Following successful rinsing with sterile 0.9% saline solution, the skin incision was closed in layers using 1-0 VICRYL® sutures (Ethicon, USA). The minipigs were able to walk and move normally after this procedure. MRI scans were performed at the indicated time points using a 3T MR scanning system (Achieva x 3.0, Philips, Germany) at the Instrument Technology Research Center, NARLabs in Taiwan. T1-weighted and T2-weighted sagittal sections were rendered using the following settings: time to repetition of 2000 ms and time to echo of 72 ms; slice thickness was 3 mm; matrix 512; TE 200; TR 3500; field of view 60 mm; number of averages 2. Tomographs DICOMs of MRI were analyzed by Osirix MD (Osirix Ltd., USA).

<Preparation of C5-24 and Scramble Peptide-Conjugated Hyaluronic Acid (HA)>

The peptide conjugated HA was synthesized as previous described with slight modification. In brief, MeHA was firstly synthesized through the reaction of methacrylic anhydride (94%, M.W. 154.17; Sigma) with 1% (wt/vol) HA (sodium hyaluronate powder, molecular weight≈110-150 kDa; Kikkoman, Japan) in deionized water at pH 8, purification via dialysis (molecular weight cutoff 6-8 kDa), followed by lyophilization. Methacrylation efficiency of the intermediate MeHA macromer was estimated by ¹H NMR. C5-24 and scrambled peptides with a cysteine residue at the C-terminal end to permit the sulfhydryl group to reacted with MeHA through Michael-Addition reaction. MeHA macromers and peptides were dissolved in triethanolamine-buffered saline (TEOA buffer, 0.2 M TEOA, 0.3 M total osmolarity, pH 8.0) and maintained at 37° C. overnight for peptide coupling. The peptide conjugated HA was subjected to dialysis in ddH₂O in M.W. 12K cut-off to remove the free-forms of peptides, TEOA, salts and MA, and further lyophilized and stored in room temperature. The peptides conjugated HA was lysed in 0.1 M acid and subjected to ¹H NMR to estimate the conjugation efficiency of peptide.

<Lubricant Performance Analysis>

Human articular cartilage samples collected from femoral condyles of cartilage were prepared for lubrication testing with a slight modification from previous publishes. Human osteoarthritic cartilage samples were sectioned from the patients who underwent total knee arthroplasty under stringent supervision by IRB committees from China Medical University Hospital (IRB number: CMUH108-REC1-046, and T-CMU-23728). Care was taken to avoid damaging the articular surface during dissection. The superficial layer of OA cartilage from individual patient was maintained intact, punch-cut to obtain a cylinder disc with diameter in 8.0 mm and 6.0 mm, respectively and only the deep layer of cartilage was cut to obtain a flat disc to glue to the metal counter-surface of the particularly designed testing modules while performing friction measurements in rheometer. Cartilage was used fresh without freezing or the addition of protease inhibitors so as not to change the surface lubrication properties. Samples were washed vigorously in PBS overnight to deplete the cartilage surface of any residual synovial fluid, after which they were separated into at least 3 groups. Cartilage discs were pre-incubated in 1 ml original HA or peptide modified HA (1% HA in PBS) for 2 hours as indicated in Results for binding of the non-modified HA or peptide modified HA with cartilage disc, followed by immersing them in 10 ml PBS in testing modules and mounting onto rheometer (HR-1, TA Instrument Ltd., USA) for friction measurements.

The rheometer was initially set to zero using standard protocol in compliance with manufacturer's instruction, and then we calculated the initial heights of the cartilage samples with an electronic caliper followed by loading the samples on the rheometer. The samples were glued with cyanoacrylate glue to the top and bottom rheometer fixtures in parallel plate configuration. Only a thin layer of glue bound to the cartilage and metal fixture surface. The 6.0 mm sample surface was positioned on top of the 8.0 mm surface. The top sample was lowered and pressed against the bottom sample until a load value of ˜0.01 N to avoid insufficient contacts between the sample surfaces, load value fluctuations and minimize the errors in height measurements. The corresponding recorded height, which was automatically sensed by rheometer, was taken for strain calculation. The instrument was programmed to record the total cartilage thickness and calculate the height for ≈14% compression. The total thickness of the human OA cartilage sample was in the range of ≈2.5-3.5 mm, which were tested in a bath of HA/PBS fluid (10 mL) covered with protecting lid to prevent desiccation. Each sample was checked for proper alignment and surface irregularity, and the experiment was performed on samples with flat surfaces. The samples were bathed in the test lubricant, compressed to 86% of their original combined height and preconditioned by rotating two revolutions in each direction at an effective sliding velocity of 0.3 mm/s, which is defined as the angular velocity times the effective radius of the annulus R_{eff}=⅔[(Ro³−Ri³)/(Ro²−Ri²)]. This preconditioning was repeated twice more, followed by a 3600 seconds stress-relaxation period to allow the pressurization of the fluid in the compressed cartilage to fully subside. The equilibrium normal stress data recording and measurement was performed for each experimental group. Lubrication testing was performed in 14 stages. The first two stages were considered negligible and used as a clearing or pre-shear stage. Stages 3, 6, 9 and 12 were performed to analyze the effect of different durations of relaxation. Samples were allowed to relax between tests for 1200, 120, 12 and 1.2 seconds. Lubrication data were recorded during stages 4-5, 7-8, 10-11 and 13-14; each stage was in a different direction of rotation and at a constant shear rate. During each test, torque (T) and axial force (N) were measured, and instantaneous measurements of μₖ, the kinetic friction coefficient, were determined from the following equation: μₖ=τ/(R_{eff}×N). Instantaneous μₖ values were averaged over the second revolution in each direction to produce an average μₖ that was used for comparison. Static friction coefficients were calculated as the instantaneous μₛ=τmax/(R_{eff}×N) at the maximal torque value found during the startup period of the test. After experimentation a central indentation due to ≈14% compression on the cartilage surface was confirmed.

<Rat MSCs Isolation and Labeling with SPIO, and Delivery Via C5-24 Peptide Conjugated HA>

Rat MSCs were isolated and expanded as previously described. Briefly, femora collected from 2 female Sprague-Dawley rats with 8 to 10 weeks of age (BioLASCO Taiwan Co Ltd, Taipei, Taiwan), and the soft tissues were detached aseptically. The bone marrow mononuclear cells were isolated by the density gradient centrifugation method and suspended in complete culture medium (CCM: α-MEM supplemented with 16.6% fetal bovine serum, 100 U/mL penicillin, 100 μg/mL streptomycin, and 2 mM L-glutamine), then seeded in culture dishes in the density of 1×10⁵/cm². Nonadherent cells were removed by washing and changing medium at 24 hours later. When cells reached sub-confluence, the cells (passage 0) were harvested for further subcultures. Then, the cells were seeded at density of 100 cells/cm² and grown in CCM with medium change twice per week. The MSCs used in this study were passage 3-4.

For MSCs labelling with superparamagnetic iron oxide nanoparticles (SPIO), 50 μg/mL of SPIO (Chemicell GmbH, Germany) was pre-mixed with 0.75 μg/mL poly-L-lysine (Sigma Aldrich, USA) in culture medium at room temperature for 1 hour. For endocytosis of SPIO nanoparticles, MSCs were seeded in 6-well plate at density of 4×10⁴/well and grown for 24 hours, followed by thoroughly washed with PBS. Then, the MSCs were collected to a microtube and incubated with 2% C5-24 peptide conjugated HA in serum-free medium in concentration of 1×10⁶ cells/200 μl at 37° C. for 30 mins. For intra-articular injection, the volume of HA encapsulated MSCs was reduced to 25 μl containing 1×10⁶ cells, and were sophisticatedly injected into a OA rat knee joint synovium capsule.

<Histological, Immunocytofluorescent and Immunohistochemical Analysis and Confocal Microscopic Observation>

Histological analysis of HA encapsulated MSCs transplantation, rats were sacrificed at post-transplantation time point as indicated in the results, whole knee joints were removed, fixed with 4% paraformaldehyde (PFA) in PBS, decalcified in 0.5 M EDTA for 2 weeks, embedded in paraffin and serial sectioned in 5 μm thickness in sagittal direction. Serial sections in the mid-zone of femoral condyle were prepared for H&E staining, Prussian blue staining and Safranin-O staining by standard protocol and observed by phase contrast microscope (Carl Zeiss). For H&E staining, de-paraffined slides were serially rehydrated, stained with Lillie Mayer haematoxylin (Sigma Aldrich, USA) for 10 mins, followed with eosin Y (Sigma Aldrich, USA) for 30 seconds, finally with serial dehydrated, cleared and mounted. For Prussian blue staining, slides preparation similar to H&E, rehydrated slides were stained with 5% potassium ferrocyanide in 10% HCl solution (Sigma Aldrich, USA) for 20 mins, countered stained with Fast Red and finalized with dehydrated, cleared and mounted with resinous gel and coverslip. For Safranin-O staining, rehydrated slides were stained with 0.05% Fast Green solution for 3 mins, followed with 0.1% Safranin-O solution for 5 mins, and finalized with cleared and mounted with resinous gel.

For confocal microscopic observation of HA encapsulated MSCs, the HA was methacrylated and conjugated with Alexa-488 fluorescent dye, prepared in 2% in PBS. MSCs were collected to a microtube, labeled with Dil3 fluorescent dye (Invitrogen, USA) according to the manufacturer's instruction and incubated with HA solution at 37° C. for 30 mins. Subsequently, dropped onto a slide and immediately observed by confocal microscope (Leica), 3D images were reconstructed by ImageJ Fiji (NIH).

<Identifying the Target Protein of the C5-24 Peptide by Affinity Trapping, Liquid Chromatography-Tandem Mass Spectrometry (LC-MS/MS) and ELISA>

To identify the binding target of C5-24 peptide chain, human OA cartilage specimens were homogenized for affinity trapping. The biotinylated C5-24 peptide in 1 mg/ml in PBS was added to the cartilage homogenate and incubated at 4° C. for 1 hour. After washing, the DTSSP solution was added to a final concentration of 2 mM for peptide-target protein cross-linking. The reaction mixture was incubated and rotated at room temperature for 30 mins. The reaction was stopped with 1 M Tris base. After lysing the chondrocytes with the first lysis buffer (1 M NaCl in 100 mM Tris acetate, pH 8.0) at 4° C. for 24 hours, the lysates were centrifuged, and the pellet was re-treated with the second lysis buffer (4 M guanidine HCl, 65 mM DTT, 10 mM EDTA in 50 mM sodium acetate, pH 5.8) at 4° C. for another 24 hours. Following centrifugation, the guanidine extracts were mixed with 100% ethanol (5:1 volume ratio) at −20° C. for 16 hours to ensure removal of the residual guanidine HCl. The target protein fraction was precipitated by centrifugation at 16,000×g and 4° C. for 45 mins, the pellet was washed with 90% ethanol, dried, and re-dissolved with 100 mM acetic acid containing 100 μg/ml pepsin. MyOne Streptavidin C1 Dynabeads (Invitrogen, Carlsbad, Calif., USA) were added to the protein lysates and mixed thoroughly for 1 hour. Immuno-magnetic separation was used to pull down the peptide-protein complexes. Finally, the purified proteins were separated by gradient sodium dodecyl sulfate-polyacrylamide gel electrophoresis (SDS-PAGE) (Bio-Rad) and silver-stained with a SilverQuest Silver Staining Kit (Invitrogen).

The stained protein bands were cut into small pieces and washed with 10 mM ammonium bicarbonate (ABC, Sigma, St Louis, Mo.) containing 50% ACN for 5 mins three times. The gel pieces were dehydrated with 100% CAN and rehydrated with 25 mM ABC (pH 8.2) solution containing 1 ng/μl trypsin (Promega, Madison, Wis.) and then incubated at 37° C. overnight. After digestion, the tryptic peptides were extracted from the gel using 1% FA in 50% ACN and dry using a centrifugal concentrator. The peptide fragments were identified by LC-MS/MS. LC-MS/MS was performed using an ion trap mass spectrometer (HCTultra PTM discovery, Bruker, Billerica, Mass.) coupled online with Ultimate 3000 nanoLC system (Dionex, Sunnyvale, Calif.). The sample was injected into a trap column (C18, 5 μm, 1 mm×5 mm, Dionex, Sunnyvale, Calif.) and separated online with a reverse phase column (Atlantis C18, 3 μm, 75 μm×150 mm, Waters, Milford, Mass.) at flow rate of 300 nl/min. Peptides were eluted with H₂O/ACN gradient from 2 to 40% of solvent B (100% ACN, 0.1% FA) in 6 mins and 40 to 70% of B in 24 mins. MS and MS/MS scan range is 400-1600 m/z and 100-2500 m/z, respectively. Protein candidates were identified by searching the Swiss Protein Database using the MASCOT (Matrix Science, London, UK) and TurboSequest search engines (Thermo Fisher Scientific, Waltham, Mass., USA), following validated by ELISA.

Firstly, ELISA plate was coated with collagen alpha-3 (VI) and collagen alpha-1 (XII) in coating buffer (0.5M NaHCO₃) at room temperature for 2 hours and blocked with 5% milk/TBST at 4° C. overnight. The biotinylated peptide was added into ELISA plate and incubated at room temperature for 1 hour. The plate was washed with PBS and the biotinylated peptide was probed with HRP-conjugated mouse anti-M13 antibody (GE Healthcare Biosciences). Binding of the biotinylated peptide to the recognized collagen alpha-3 (VI) or collagen alpha-1 (XII) was detected by HRP-conjugated streptavidin (Thermo Pierce Biotechnology Scientific). The plate was washed with PBS and subsequently incubated with peroxidase substrate ophenylenediamine dihydrochloride (OPD; Sigma). The reaction was terminated by 3 N HCl, and the absorbance at 490 nm was measured with an ELISA reader.

<Homology Modeling of C5-24 Peptide Docking Target>

Molecular modeling to further confirm the binding target of selected phage clones on cartilage tissue was performed by Dassault Systems (BIOVIA, Discovery Studio Modeling Environment, Release 2019, San Diego, USA) in compliance with the developer's instruction. Briefly, the standard sequence code of human, mouse, and pig ColXII which retrieved from Uniprot Database were Q99715, Q60847, F1 RQIO, individually. Three different parts of human ColXII homology models were built using MODELER based on the templates (PDB code: 1 FNF, 2B2X, 2UUR) from BLAST result. The length of first human ColXII model was from L1385 to S2285, with 30% identity to the template 1 FNF, which indicated the fibronectin structure and could be used to model establishment due to the highly conserved structural topology. The second and third human ColXII models were from K2321 to L2513 and S2506 to P2724 with 31% and 36% sequence identity with template 2B2X and 2UUR, individually. All of the homology models were firstly checked by PDF total energy, DOPE (Discrete Optimized Protein Energy) and verify score, Ramachandran plot and refined the structure to obtain the reasonable backbone and sidechain conformation. The most representative protein templates were used to predict the binding sites and poses with C5-24 and C5-91 peptide chains due to the most promising results in IHC. Subsequently, Protein-peptide docking using ZDOCK was performed for searching the potential binding region. The Z_Dock score and E_R_Dock score were used to validate the docking capability and exactitude between peptides and target protein templates.

<Statistical Analysis>

Data are presented as mean±SD, statistical comparisons were performed by Student's t-test or one-way analysis of variance (ANOVA) and p values<0.05 were considered significant. All calculations were performed using Statistics Analysis System (SAS) licensed to China Medical University. All in vivo data are representative of at least 3 independent experiments as indicated.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure covers modifications and variations of this disclosure provided they fall within the scope of the following claims.

1. An oligopeptide, comprising:
an amino acid sequence comprising a binding motif, wherein:
the binding motif is represented by Formula (i):
WX₁PX₂W (i),
wherein W is tryptophan, P is proline, X₁ and X₂ are respectively an amino acid, and X₁ and X₂ are identical or different from each other; or
the binding motif is represented by Formula (ii):
DTH (ii),
wherein D is aspartic acid, T is threonine, and H is histidine.

2.-3. (canceled)

4. The oligopeptide of claim 1, wherein the amino acid sequence has at least 90% identity with at least one of the full-length amino acid sequences of SEQ ID NO. 1, SEQ ID NO. 2, SEQ ID NO. 3 or SEQ ID NO. 4.

5. (canceled)

6. The oligopeptide of claim 4, wherein the amino acid sequence is identical to at least one of the full-length amino acid sequences of SEQ ID NO. 1, SEQ ID NO. 2, SEQ ID NO. 3 or SEQ ID NO. 4.

7.-10. (canceled)

11. The oligopeptide of claim 1, wherein a binding target of the oligopeptide is collagen XII, and the oligopeptide has a binding specificity for a cartilage tissue suffered from osteoarthritis.

12. (canceled)

13. A testing kit, comprising:
the oligopeptide of any one of claims 1, 4, 6 and 11.

14. The testing kit of claim 13, wherein the oligopeptide is bound to a superparamagnetic iron oxide (SPIO) or an image developer.

15. A medical composition, comprising:
the oligopeptide of any one of claims 1, 4, 6 and 11; and
a treatment molecule or a stem cell binding to the oligopeptide.

16. The medical composition of claim 15, wherein the treatment molecule is an osteoarthritis treating drug, an intervertebral disease treating drug, an eye disease treating drug, a hyaluronic acid, a cartilage growth factor or a composition thereof, and stem cell is mesenchymal stem cell (MSC).

17.-19. (canceled)
